(19) United States
(12) Patent Application Publication
(10) Pub. No.: US 2015/0297495 A1
(43) Pub. Date: Oct. 22, 2015

(54) A COLOR COSMETIC COMPOSITION WITH INCREASED COLOR INTENSITY

(71) Applicants: Praddhyumana Patel, Grayslake, IL (US); Lilavati Patel, Edison, NJ (US); Dhaval Patel, Basking Ridge, NJ (US)

(72) Inventors: Praddhyumana Patel, Grayslake, IL (US); Lilavati Patel, Edison, NJ (US); Dhaval Patel, Basking Ridge, NJ (US)

(73) Assignee: L'OREAL, Paris (FR)

(21) Appl. No.: 14/420,201

(22) PCT Filed: Aug. 10, 2012
(86) PCT No.: PCT/US2012/050298

§ 371 Date: Feb. 6, 2015

(51) Int. Cl. A61K 8/81; A61K 8/19; A61Q 1/10; A61K 8/73

(52) U.S. Cl. CPC A61K 8/8147; A61K 8/732; A61K 8/19; A61Q 1/10; A61K 2800/43

(57) ABSTRACT

A cosmetic composition for increasing color intensity that has at least one colorant, at least one anionic polymer, at least one starch, and optionally, at least one mattifying agent, optionally, at least one auxiliary ingredient, wherein the at least one anionic polymer and the at least one starch form hydrogen bonds with the at least one colorant. In addition, a method of increasing color intensity of a color cosmetic composition, wherein the method contains the following steps: (a) providing the cosmetic composition for increasing color intensity; (b) increasing an amount of the at least one pigment to a range from about 5% to about 15% by weight; (c) reducing the at least one mattifying agent to a range from about 1% to about 25% by weight; and (d) maintaining about 2% to about 8% by weight of the at least one anionic polymer and about 0.5% to about 4% by weight of the modified starch, wherein the weight percentages are based on the total weight of the composition, and the composition exhibits increased color intensity. Further, a mascara containing the cosmetic composition for increasing color intensity.

This application is a national stage application (under 35 U.S.C. §371) of PCT/US12/050298, filed Aug. 10, 2012.

### BACKGROUND OF THE INVENTION

Consumers are typically drawn to cosmetic compositions for a variety of reasons, including the intensity of the colors provided by the compositions. For example, color formulations in cosmetics, including those for mascaras, have utilized carbon black to provide, inter alia, an intense and deep black color. Regulatory groups have, however, questioned the health and environmental impact of carbon black. For instance, the California Office of Environmental Health Hazard Assessment has listed carbon black (airborne, unbound particles of respirable size) as a substance known to cause cancer. Therefore, the need exists for an alternative and/or replacement for carbon black that can provide the color intensity that is desired by consumers.

To increase the color intensity of cosmetic formulations, particularly for mascaras, several approaches have been taken. For instance, using a base for layering with the color formulation may increase color intensity. On the other hand, specialty silicones may be added to the color formulations to bring forth more vibrant and richer colors. Another approach is to incorporate into the color formulation an esterification product between a C₁₂-C₁₄ fatty alcohol and a dimer or trimer of C₁₄-C₂₄ fatty acid having 2 to 4 unsaturation as described in U.S. Patent Application Publication US 2007/0077217. Nevertheless, further improvement in color intensity can be achieved.

Additional approaches to enhance color intensity of cosmetic formulation include the use of different types of pigments. However, no pigment has been found to be capable of matching the color intensity of carbon black.

As such, it has been surprisingly and unexpectedly discovered that cosmetic compositions comprising at least one colorant, at least one anionic polymer and at least one starch provide increased color intensity for a variety of cosmetic products including, for example, mascaras and eyeliners.

It has also been discovered that the cosmetic compositions are able to provide an alternative and/or replacement for carbon black that provides the color intensity of carbon black, as well as additional colors. It has even further been discovered that the cosmetic compositions according to the present invention can be used to provide mascara and further consumer products with increased color intensity.

### BRIEF SUMMARY OF THE INVENTION

The present invention is directed to a cosmetic composition for increasing color intensity, comprising at least one colorant, at least one anionic polymer, at least one starch, and optionally, at least one mattifying agent, and optionally, at least one auxiliary ingredient, wherein the at least one anionic polymer and the at least one starch form hydrogen bonds with the at least one colorant.

According to one preferred embodiment, the cosmetic composition for increasing color intensity comprises about 5% to about 20% by weight of the at least one colorant, about 1% to about 12% by weight of the at least one anionic polymer, and about 0.5% to about 5% by weight of the starch, wherein the weight percentages are based on the total weight of the composition.

According to another preferred embodiment, the cosmetic composition for increasing color intensity comprises about 5% to about 15% by weight of the at least one colorant, about 2% to about 8% by weight of the at least one anionic polymer, and about 0.5% to about 4% by weight of the starch, wherein the weight percentages are based on the total weight of the composition.

According to yet another preferred embodiment, the cosmetic composition for increasing color intensity comprises about 5% to about 20% by weight of the at least one colorant, the cosmetic for increasing color intensity comprises about 1% to about 12% by weight of the at least one anionic polymer, about 0.5% to about 5% by weight at least one starch, and about 1% to about 35% by weight at least one mattifying agent, about 48% to about 91.5% by weight of the at least one auxiliary ingredient, wherein the weight percentages are based on the total weight of the composition.

According to still yet another preferred embodiment, the cosmetic composition for increasing color intensity comprises about 5% to about 15% by weight of the at least one colorant, the cosmetic for increasing color intensity comprises about 2% to about 8% by weight of the at least one anionic polymer, about 0.5% to about 4% by weight at least one starch, and about 1% to about 25% by weight at least one mattifying agent, about 25% to about 75% by weight of the at least one auxiliary ingredient, wherein the weight percentages are based on the total weight of the composition.

In yet another preferred embodiment, the cosmetic composition for increasing color intensity comprises at least one colorant, wherein the at least one colorant is iron oxide; at least one anionic polymer, wherein the at least one anionic polymer is ethylene/acrylic acid copolymer; and at least one starch, wherein the at least one starch is hydrolyzed corn starch, wherein the at least one anionic polymer and the at least one starch forms hydrogen bonds to aid in pigment dispersion in the composition and to increase the viscosity of the composition.

In yet another preferred embodiment, the cosmetic composition for increasing color intensity comprises at least one colorant, wherein the at least one colorant is iron oxide; at least one anionic polymer, wherein the at least one anionic polymer is ethylene/acrylic acid copolymer; and at least one starch, wherein the at least one starch is hydrolyzed corn starch; at least one mattifying agent, wherein the at least one mattifying agent is wax; at least one auxiliary ingredient, wherein the at least one auxiliary ingredient is film former; wherein the at least one anionic polymer and the at least one starch forms hydrogen bonds to aid in pigment dispersion in the composition and to increase the viscosity of the composition.

The present invention is also directed to a method of increasing color intensity of a color cosmetic composition. Said method comprises:

(a) providing the cosmetic composition for increasing color intensity;

(b) increasing an amount of the at least one pigment to a range from about 5% to about 15% by weight;

(c) reducing the at least one mattifying agent to a range from about 1% to about 25% by weight; and

(d) maintaining about 2% to about 8% by weight of the at least one anionic polymer and about 0.5% to about 4% by weight of the modified starch,

wherein the weight percentages are based on the total weight of the composition, and the composition exhibits increased color intensity.

The present invention is even further directed to a mascara comprising at least one colorant, at least one anionic polymer, at least one starch, and optionally, at least one mattifying agent, optionally, at least one auxiliary ingredient, wherein the at least one anionic polymer and the at least one starch form hydrogen bonds with the at least one colorant.

The present invention can be further used in a wide variety of cosmetic products, including, but not limited to: eyeliners, eye shadows, lipsticks and foundations.

### DETAILED DESCRIPTION OF THE INVENTION

Other than in the operating examples, or where otherwise indicated, all numbers expressing quantities of ingredients or reaction conditions are to be understood as being modified in all instances by the term “about.”

The term “comprising” (and its grammatical variations) as used herein is used in the inclusive sense of “having” or “including” and not in the exclusive sense of “consisting only of.” The terms “a” and “the” as used herein are understood to encompass the plural as well as the singular.

The term “at least one”, as used herein, means one or more, and thus the term includes individual components as well as mixtures or combinations.

The composition and methods of the present invention can comprise, consist of, or consist essentially of the essential elements and limitations of the invention described herein, as well as any additional or optional ingredients, components, or limitations described herein or otherwise useful ingredients found in cosmetic compositions.

### Colorant

The cosmetic composition for increasing color intensity comprises at least one colorant. In accordance with the present invention, suitable colorants include, for example: pigments, dyes, lakes, and mixtures thereof as well as other colorants that would be readily apparent to one skilled in the art.

In one embodiment, the colorant that can be utilized for the composition of the invention includes iron oxides. For example, such iron oxides may include Fe₃O₄ and Fe₂O₃. Further, additional color pigments that can be utilized include, for example: chromium oxide, titanium dioxide, and ultramarines.

In another embodiment, the colorant that can be utilized for the composition of the invention includes carbon black (Colour Index International, PBK-7).

As a component in the cosmetic composition for increasing color intensity, the at least one colorant exists in a range. In one embodiment, the at least one colorant is present in the composition in the range of about 5% to about 20% by weight, wherein the weight percentages are based on the total weight of the composition. In another embodiment, the at least one colorant is present in the composition in the range of about 5% to about 15% by weight, wherein the weight percentages are based on the total weight of the composition.

### Anionic Polymer

The cosmetic composition for increasing color intensity comprises at least one anionic polymer. In accordance with the present invention, suitable anionic polymers include, for example: ethylene/acrylic acid copolymers.

The anionic polymer can provide hydrogen bond formation with colorants that assist in the dispersion of the color pigments. For instance, a useful hydrogen bond forming anionic copolymer is ethylene/acrylic acid copolymer (EAA). The EAA copolymer has the combined structural features of an ethylene group (E), which is non-polar, water resistant and flexible, and an acrylic acid group (AA), which is polar and capable of crosslinking. As a whole, the EAA copolymer, offers stable separation through electrostatic repulsion of the negatively-charged carboxylate and while allowing interaction with other EAA copolymer molecules and the color pigments. As a result, color pigments are effectively dispersed.

As a component in the cosmetic composition for increasing color intensity, the at least one anionic polymer exists in a range. In one embodiment, the at least one anionic polymer is present in the composition in the range of about 1% to about 12% by weight, wherein the weight percentages are based on the total weight of the composition. In another embodiment, the at least one anionic polymer is present in the composition in the range of about 2% to about 8% by weight, wherein the weight percentages are based on the total weight of the composition.

### Starch

The cosmetic composition for increasing color intensity comprises at least one starch. In accordance with the present invention, suitable starches include, for example: modified starches, rice starch, and potato starch. One particularly preferred starch according to the present invention is hydrolyzed corn starch.

In general, the at least one starch may serve various purposes, such as being surfactants, emulsifiers and thickening agents. The starch may work in conjunction with the anionic polymer to form hydrogen bonds and thereby further provide optimal pigment dispersion and optimal viscosity.

As a component in the cosmetic composition for increasing color intensity, the at least one starch exists in a range. In one embodiment, the at least one starch is present in the composition in the range of about 0.5% to about 5% by weight, wherein the weight percentages are based on the total weight of the composition. In another embodiment, the at least one starch is present in the composition in the range of about 0.5% to about 4% by weight, wherein the weight percentages are based on the total weight of the composition.

### Mattifying Agent

The cosmetic composition for increasing color intensity optionally comprises at least one mattifying agent. In accordance with the present invention, suitable starches include, for example: waxes, talc, and silica. Particularly preferred mattifying agents include beeswax and copernicia cerifera (carbauba) wax.

As a component in the cosmetic composition for increasing color intensity, the at least one mattifying agent exists in a range. In one embodiment, the at least one mattifying agent is present in the composition in the range of about 1% to about 35% by weight, wherein the weight percentages are based on the total weight of the composition. In another embodiment, the at least one mattifying agent is present in the composition in the range of about 1% to about 25% by weight, wherein the weight percentages are based on the total weight of the composition.

### Auxiliary Ingredient(s)

The cosmetic composition for increasing color intensity optionally comprises at least one auxiliary ingredient. In accordance with the present invention, suitable auxiliary ingredients include, for example: film formers, methicones, solvents, alcohols, preservatives, emulsifiers, thickeners, thinning agents, stabilizers, fragrances, extracts, binders, vitamins, silicones, plasticizers, and mixtures thereof. Further additional auxiliary ingredients would be readily recognized by one of ordinary skill in the art.

As a component in the cosmetic composition for increasing color intensity, the at least one auxiliary ingredient exists in a range. In one embodiment, the at least one auxiliary ingredient is present in the composition in the range of about 48% to about 91.5% by weight, wherein the weight percentages are based on the total weight of the composition. In another embodiment, the at least one auxiliary ingredient is present in the composition in the range of about 25% to about 75% by weight, wherein the weight percentages are based on the total weight of the composition.

The viscosity of the cosmetic composition for increasing color intensity can be controlled by using the combination of components described herein. The viscosity can be controlled by varying the amount of one component in the composition with respect to the other components. For example, if the at least one mattifying agent is a wax, then by lowering the amount of the at least one mattifying agent that is a wax in the composition, the viscosity of the overall composition would increase. The composition preferably has a viscosity in the range of 10 to 85 UD. More preferably, the composition has a viscosity in the range of 25 to 60 UD.

### EXAMPLES

The invention is illustrated in greater details in the following examples, which are exemplary and therefore not limiting.

### Example 1

### Exemplary Formulation 1

Washable Mascara
683910

| US Name | Concentration |
|---|---|
| WATER | 42.35 |
| ACRYLATES COPOLYMER | 0.01 |
| PHENOXYETHANOL | 0.5 |
| SODIUM DEHYDROACETATE | 0.2 |
| SIMETHICONE | 0.19 |
| GLYCERIN | 2 |
| DISODIUM EDTA | 0.1 |
| ACACIA SENEGAL GUM | 3.39 |
| HYDROXYETHYLCELLULOSE | 0.89 |
| IRON OXIDES | 7.14 |
| POTASSIUM CETYL PHOSPHATE | 8.27 |
| BEESWAX | 4.37 |
| COPERNICIA CERIFERA (CARNAUBA) WAX | 3.45 |
| PARAFFIN | 13.82 |
| HYDROGENATED JOJOBA OIL | 0.2 |
| HYDROGENATED PALM OIL | 0.2 |
| STEARETH-20 | 0.5 |
| CETYL ALCOHOL | 2 |
| ETHYLENE/ACRYLIC ACID COPOLYMER | 5.5 |
| SODIUM POLYMETHACRYLATE | 1 |
| WATER | 2.4 |
| POLYQUATERNIUM-10 | 0.1 |
| PEG/PPG-17/18 DIMETHICONE | 0.5 |
| WATER | 0.5 |
| PANTHENOL | 0.01 |
| ASCORBYL PALMITATE | 0.01 |
| SACCHARUM OFFICINARUM (SUGAR CANE) | 0.1 |
| EXTRACT (and) CITRUS MEDICA | |
| LIMONUM (LEMON) PEEL EXTRACT (and) | |
| PYRUS MALUS (APPLE) FRUIT EXTRACT | |
| (and) CAMELLIA SINENSIS LEAF EXTRACT | |
| CAPRYLYL GLYCOL | 0.3 |
| Total | 100 |

### Example 2

### Exemplary Formulation 2

Washable Mascara
683952

| US Name | Concentration |
|---|---|
| WATER | 45.74 |
| PHENOXYETHANOL | 0.5 |
| SODIUM DEHYDROACETATE | 0.2 |
| GLYCERIN | 2 |
| DISODIUM EDTA | 0.1 |
| HYDROLYZED CORN STARCH | 2 |
| ACACIA SENEGAL GUM | 3.39 |
| HYDROXYETHYLCELLULOSE | 0.89 |
| CAPRYLYL GLYCOL | 0.3 |
| IRON OXIDES | 10 |
| POTASSIUM CETYL PHOSPHATE | 8.27 |
| BEESWAX | 3.14 |
| COPERNICIA CERIFERA (CARNAUBA) WAX | 2.48 |
| PARAFFIN | 9.94 |
| HYDROGENATED JOJOBA OIL | 0.14 |
| HYDROGENATED PALM OIL | 0.14 |
| STEARETH-20 | 0.36 |
| CETYL ALCOHOL | 1.4 |
| ETHYLENE/ACRYLIC ACID COPOLYMER | 4.5 |
| SODIUM POLYMETHACRYLATE | 1 |
| WATER | 2.4 |
| POLYQUATERNIUM-10 | 0.1 |
| PEG/PPG-17/18 DIMETHICONE | 0.5 |
| WATER | 0.5 |
| PANTHENOL | 0.01 |
| Total | 100 |

### Example 3

### Color Intensity

L*a*b* color space was measured using a Konica Minolta CR-400 Colorimeter. The instrument settings were: reflectance measurement, 2 degree observer, and C illuminant). Samples of an embodiment of the present invention were measured in both the wet state and as a dried film. The dried film was allowed to dry for 24 hours before measurement. Twelve measurements were taken per sample and averaged together. The significance level was set at p value <0.05. For the L*a*b* system, “L” is an indicator of how black (when L=0) or white (when L=100) the color is. The value “a” is an indicator of how red (+a) or green (−a) the color is. The value “b” is an indicator of how yellow (+b) or blue (−b) the color is.

A comparison of two mascaras is presented in the tables below. Sample 1 is the Exemplary Formulation 2 discussed in Example 2. Sample 2 is similar to the Exemplary Formulation 2 discussed in Example 2 except the hydrolyzed corn starch and ethylene/acrylic acid copolymer was removed.

Wet

| Formula | L* | a* | b* | C* | h | ΔE | ΔE₀₀ |
|---|---|---|---|---|---|---|---|
| Sample 1 | 22.70 | 0.94 | −0.83 | 1.25 | 318.61 | 0.35 | 0.32 |
| | (±0.35) | (±0.03) | (±0.06) | (±0.06) | (±1.74) | | |
| Sample 2 | 22.90 | 0.87 | −1.11 | 1.41 | 308.10 | | |
| | (±1.57) | (±0.05) | (±0.03) | (±0.05) | (±1.20) | | |
| p value | 0.089 | 0.001 | <0.001 | <0.001 | <0.001 | | |

Dry

| Formula | L* | a* | b* | C* | h | ΔE | ΔE₀₀ |
|---|---|---|---|---|---|---|---|
| Sample 1 | 23.26 | 0.83 | −0.36 | 0.91 | 336.93 | 1.70 | 1.23 |
| | (±0.07) | (±0.04) | (±0.02) | (±0.04) | (±1.06) | | |
| Sample 2 | 24.96 | 0.82 | −0.36 | 0.89 | 336.07 | | |
| | (±0.03) | (±0.03) | (±0.02) | (±0.03) | (±0.75) | | |
| p value | <0.001 | 0.232 | 0.401 | 0.399 | 0.032 | | |

According to the data presented, the a*, b*, C* and h of the wet formulation of sample 1 were significantly different than the wet formulation of sample 2. Further, the L* of the dry formulation of sample 1 was significantly different than the wet formulation of sample 2. The L* value, in particular, indicates that the sample 1 formulation had more blackness than sample 2.

### Example 4

### Color Intensity

Further studies were conducted to measure the “blackness” of mascara samples using the L*a*b* system and the Munsell hue-value-chroma scale (L). The L*a*b* was previously discussed in Example 3. In the Munsell scale, lightness is termed value and measured using a 0-8 scale, with 0 the darkest and 8 the lightest.

In this study, a hand-held colorimeter was used in conjunction with a granular sample holder to conduct blackness measurements using the L*a*b* system and the Munsell hue-value-chroma scale (L). The samples that were compared were the exemplary formula of Example 2 and a sample of mascara according to the formulation presented in the table below.

Washable Mascara
683659.1

| US Name | Concentration |
|---|---|
| WATER | 44.77 |
| PHENOXYETHANOL | 0.66 |
| SIMETHICONE | 0.13 |
| METHYLPARABEN | 0.29 |
| SODIUM DEHYDROACETATE | 0.2 |
| GLYCERIN | 2 |
| DISODIUM EDTA | 0.1 |
| ACACIA SENEGAL GUM | 3.39 |
| HYDROXYETHYLCELLULOSE | 0.89 |
| IRON OXIDES | 0 |
| BLACK 2 | 10 |
| POTASSIUM CETYL PHOSPHATE | 8.27 |
| BEESWAX | 4.37 |
| COPERNICIA CERIFERA (CARNAUBA) WAX | 3.45 |
| PARAFFIN | 13.82 |
| HYDROGENATED JOJOBA OIL | 0.2 |
| HYDROGENATED PALM OIL | 0.2 |
| STEARETH-20 | 0.5 |
| CETYL ALCOHOL | 2 |
| ETHYLPARABEN | 0.15 |
| SODIUM POLYMETHACRYLATE | 1 |
| WATER | 2.4 |
| POLYQUATERNIUM-10 | 0.1 |
| PEG/PPG-17/18 DIMETHICONE | 0.5 |
| WATER | 0.5 |
| PANTHENOL | 0.01 |
| SOLUBLE COLLAGEN | 0.1 |
| Total | 100 |

For each mascara, six (6) samples were tested for the L*a*b* and Munsell L values in bulk phase measured with the Konica-Minolta CR400. Samples were contained within the sample holder by the transparent window through which the measurements were made. The acquisition software was set to illuminate the sample with three pulses from the source light, with ten seconds between pulses. This allows time to rotate the samples approximately 120° between pulse/acquisition cycles. Observer and illuminant were set to 2° and C, respectively. The three data points per sample were averaged and the six individual sample averages were, in turn, averaged. The error is expressed in terms of the standard deviation of the six sample values. The results of the study are presented in the table below.

| | Color Cosmetic Formulation | | | Color Cosmetic Formulation | | |
|---|---|---|---|---|---|---|
| | with Carbon Black | | | without Carbon Black | | |
| L*a*b* | L* | a* | b* | L* | a* | b* |
| Sample 1 | 21.28 | 0.41 | 0.08 | 18.51 | 1.43 | −0.05 |
| Sample 2 | 20.92 | 0.45 | 0.03 | 18.13 | 1.47 | 0.27 |
| Sample 3 | 20.55 | 0.51 | −0.05 | 18.24 | 1.38 | 0.38 |
| Sample 4 | 20.16 | 0.52 | −0.07 | 18.21 | 1.46 | 0.10 |
| Sample 5 | 20.15 | 0.57 | −0.06 | 18.17 | 1.43 | 0.44 |
| Sample 6 | 20.18 | 0.54 | −0.02 | 17.82 | 1.48 | 0.35 |
| Average | 20.54 | 0.50 | −0.01 | 18.18 | 1.44 | 0.25 |
| St. Dev. | 0.47 | 0.06 | 0.06 | 0.22 | 0.04 | 0.19 |
| P | <0.001 | <0.001 | 0.008723 | | | |

According to the data presented, the exemplary formula of Example 2, which does not include carbon black, has a smaller L value, a larger a* value and a larger b* value than 683659.1 Formulation, which includes carbon black nanoparticles. Therefore, the mascara according to the invention is darker, greener and more yellow than the formulation comprising carbon black.

| | Color | |
|---|---|---|
| | Cosmetic Formulation | Color Cosmetic Formulation |
| | with Carbon Black | without Carbon Black |
| Munsell L Values | Nanoparticles | Nanoparticles |
| Sample 1 | 2.07 | 1.80 |
| Sample 2 | 1.96 | 1.76 |
| Sample 3 | 1.96 | 1.77 |
| Sample 4 | 1.99 | 1.76 |
| Sample 5 | 1.95 | 1.76 |
| Sample 6 | 1.96 | 1.73 |
| Average | 1.98 | 1.76 |
| St. Dev. | 0.04 | 0.02 |
| P | <0.001 | <0.001 |

According to the data presented, the exemplary formula of Example 2, which does not include carbon black nanoparticles, has a smaller L value than 683659.1 Formulation, which includes carbon black nanoparticles. Therefore, the mascara according to the invention is darker than the formulation comprising carbon black.

### Example 5

### Expert Panel Testing

Testing was done to evaluate two mascara samples for application, visual, aesthetic, and tactile attributes to determine if there are noticeable differences detected by an expert panel. One mascara sample provided to the expert panel was the exemplary formula of Example 2. The other mascara sample was 683573.1 Formulation, the formulation of which is presented in the table below.

Washable Mascara
683573.1

| US Name | Concentration |
|---|---|
| WATER | 47.93 |
| PHENOXYETHANOL | 0.5 |
| SIMETHICONE | 0.13 |
| GLYCERIN | 2 |
| DISODIUM EDTA | 0.1 |
| ACACIA SENEGAL GUM | 3.39 |
| HYDROXYETHYLCELLULOSE | 0.89 |
| IRON OXIDES | 7.14 |
| POTASSIUM CETYL PHOSPHATE | 8.27 |
| BEESWAX | 4.37 |
| COPERNICIA CERIFERA (CARNAUBA) WAX | 3.45 |
| PARAFFIN | 13.82 |
| HYDROGENATED JOJOBA OIL | 0.2 |
| HYDROGENATED PALM OIL | 0.2 |
| STEARETH-20 | 0.5 |
| CETYL ALCOHOL | 2 |
| SODIUM POLYMETHACRYLATE | 1 |
| WATER | 2.4 |
| POLYQUATERNIUM-10 | 0.1 |
| PEG/PPG-17/18 DIMETHICONE | 0.5 |
| WATER | 0.5 |
| PANTHENOL | 0.01 |
| PHENYL ETHYL ALCOHOL | 0.5 |
| SOLUBLE COLLAGEN | 0.1 |
| Total | 100 |

Ten sensory panelists trained in the application and evaluation of mascara products participated in the study on nonconsecutive days. Two samples were evaluated each day using a sequential monadic test design. The mascara was applied to the eye lashes using a standard protocol. Samples were rated using a 15-point universal intensity scale (0=none/15=a lot), using a standard ballot for mascara. The mascaras were evaluated during application to the eye lashes, and immediately after application. After initially rated, the panelists input data into SIMS direct data entry system. The evaluations were performed in duplicate. Mascaras were also evaluated after 8 and 12 hours of wear. A summary of the results is presented in the tables below.

TABLE
EVALUATION Mean Scores at Application

| Application Attributes | | | Visual Attributes | | | Aesthetic and Tactile Attributes | | |
|---|---|---|---|---|---|---|---|---|
| | Sample | | | Sample | | | Sample | |
| Sample | 1 | 683573.1 | Sample | 1 | 683573.1 | Sample | 1 | 683573.1 |
| Fragrance | 11.0b | 12.4a | Evenness | 11.1 | 11.5 | Heaviness | 1.3a | 0.9b |
| Intensity | | | of product | | | Stickiness | 0 | 0 |
| Shininess of | 12.6a | 12.1b | deposit | | | Added | 2.5 | 2.3 |
| Product on brush | | | Evenness | 11.3 | 11.5 | Stiffness | | |
| Intensity of | 13.0a | 11.8b | of lash | | | Dryness | 14.7 | 14.6 |
| Product on brush | | | distribution | | | of Lashes | | |
| Amount of | 9.9b | 10.6a | Added | 11.1a | 10.4b | | | |
| Product on brush | | | length | | | | | |
| Globs on brush | 2.3b | 3.6a | Added | 10.4a | 9.8b | | | |
| Immediate | 7.8a | 7.3b | thickness | | | | | |
| Product Deposit | | | Added curl | 9.7a | 9.2b | | | |
| | 12.7a | 11.9b | Spiking | 13.6 | 13.4 | | | |
| | 7.5 | 7.5 | Separation | 1.7 | 1.8 | | | |
| | 2.7 | 2 | Type of | 9.5 | 9.4 | | | |
| | | | spiking | | | | | |
| | | | Balls on | 2.6a | 2.0b | | | |
| | | | lashes | | | | | |
| | | | Flaking | 0 | 0 | | | |
| | | | Color | 12.6a | 11.7b | | | |
| | | | Intensity | | | | | |
| | | | Lashes | 0.3 | 0.3 | | | |
| | | | looking dry | | | | | |

TABLE
Evaluation Mean Scores after 8 Hours

Sample

| | |
|---|---|
| Sample 1 | 683573.1 |

Visual Attributes

| | | |
|---|---|---|
| Amount | 14.1 | 13.9 |
| Product | | |
| Remaining | | |
| Smudging- | 0.3 | 0.2 |
| Smearing | | |
| transfer to | 0.8 | 0.6 |
| skin | | |
| Evenness | 10.7 | 11.2 |
| of product | | |
| remaining | | |
| Evenness | 11.1 | 11.4 |
| of lash | | |
| distribution | | |
| Added | 10.8a | 10.2b |
| length | | |
| Added | 10.1a | 9.8b |
| thickness | | |
| Added curl | 9.4a | 8.9b |
| Spiking | 13.5 | 13.4 |
| Separation | 1.8 | 1.9 |
| Type of | 9.5 | 9.2 |
| spiking | | |
| Balls on | 2 | 1.6 |
| lashes | | |
| Flaking | 0.5 | 0.3 |
| Color | 12.2a | 11.6b |
| Intensity | | |
| Lashes | 0 | 0 |
| looking dry | | |

Aesthetic and Tactile Attributes

| | | |
|---|---|---|
| Heaviness | 0 | 0 |
| Stickiness | 0 | 0 |
| Added | 2.6 | 2.3 |
| Stiffness | | |

TABLE
Evaluation Mean Scores after 12 Hours

Sample

| | |
|---|---|
| Sample 1 | 683573.1 |

Visual Attributes

| | | |
|---|---|---|
| Amount | 13.6a | 13.2b |
| Product | | |
| Remaining | | |
| Smudging- | 0.6 | 0.4 |
| Smearing | | |
| Transfer to | 0.8 | 0.6 |
| skin | | |
| Evenness | 10.4 | 10.9 |
| of product | | |
| remaining | | |
| Evenness | 10.8 | 11 |
| of lash | | |
| distribution | | |
| Added | 10.5a | 9.9b |
| length | | |
| Added | 9.9 | 9.6 |
| thickness | | |
| Added curl | 9.2a | 8.6b |
| Spiking | 13.4 | 13.3 |
| Separation | 1.8 | 1.9 |
| Type of | 9.4 | 9.1 |
| spiking | | |
| Balls on | 1.4 | 1.2 |
| lashes | | |
| Flaking | 0.6 | 1 |
| Color | 11.8a | 11.3b |
| Intensity | | |
| Lashes | 0 | 0 |
| looking dry | | |

Aesthetic and Tactile Attributes

| | | |
|---|---|---|
| Heaviness | 0 | 0 |
| Stickiness | 0 | 0 |
| Added | 2.5 | 2.3 |
| Stiffness | | |

Overall, significant differences were found between the two products at application, as well as at 8 and 12 hours of wear. Prior to the application, the 683573.1 Formulation had significantly more fragrance intensity, amount of product on the brush and globs on the brush than the exemplary formula of Example 2. Moreover, the exemplary formula of Example 2 had significantly more shininess of product on the brush and intensity on the brush than the 683573.1 Formulation.

During application, the exemplary formula of Example 2 had significantly more of the following attributes than the 683573.1 Formulation: immediate product deposit, build ability, added length, added thickness, added curl, balls on lashes, color intensity, and heaviness.

After both 8 and 12 hours of wear, the exemplary formula of Example 2 had significantly more of the following attributes than the 683573.1 Formulation: added length, added curl, and color intensity. After 8 hours of wear, the exemplary formula of Example 2 had significantly more added thickness than the 683573.1 Formulation. After 12 hours of wear, the exemplary formula of Example 2 had significantly more product remaining on the lashes than the 683573.1 Formulation.

The foregoing description illustrates and describes the invention. Additionally, the foregoing description shows and describes only the preferred embodiment(s) but, as mentioned above, it is to be understood that it is capable to use in various other combinations, modifications, and environments and is capable of changes or modifications within the scope of the invention concepts as expressed herein, commensurate with the above teachings and/or skill or knowledge of the relevant art. The embodiments described herein above are further intended to explain best modes known by the applicants and to enable others skilled in the art to utilize the disclosure in such, or other, embodiments and with the various modifications required by the particular applications or uses thereof. Accordingly, the description is not intended to limit the invention to the form disclosed herein. Also, it is intended that the appended claims be construed to include alternative embodiments.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent applications incorporated herein by reference, the present disclosure controls.

What is claimed:

1. A cosmetic composition for increasing color intensity, comprising:
at least one colorant,
at least one anionic polymer,
at least one starch, and
optionally, at least one mattifying agent,
optionally, at least one auxiliary ingredient,
wherein the at least one anionic polymer and the at least one starch form hydrogen bonds with the at least one colorant.

2. The composition according to claim 1, wherein the at least one colorant is selected from the group consisting of iron oxides, dyes, pigments, lakes, carbon black, and mixtures thereof.

3. The composition according to claim 1, wherein the at least one anionic polymer comprises ethylene/acrylic acid copolymers.

4. The composition according to claim 1, wherein the at least one starch is selected from the group consisting of hydrolyzed corn starch, rice starch, potato starch, and mixtures thereof.

5. The composition according to claim 1, further comprising at least one mattifying agent, wherein the at least one mattifying agent is selected from the group consisting of waxes, talc, silica, and mixtures thereof.

6. The composition according to claim 1, further comprising at least one auxiliary ingredient, wherein the at least one auxiliary ingredient is selected from the group consisting of film formers, methicones, solvents, alcohols, preservatives, emulsifiers, thickeners, thinning agents, stabilizers, fragrances, extracts, binders, vitamins, silicones, plasticizers, and mixtures thereof.

7. The composition according to claim 1, comprising:
about 5% to about 20% by weight of the at least one colorant,
about 1% to about 12% by weight of the at least one anionic polymer, and
about 0.5% to about 5% by weight of the starch,
wherein the weight percentages are based on the total weight of the composition.

8. The composition according to claim 7, comprising:
about 5% to about 15% by weight of the at least one colorant,
about 2% to about 8% by weight of the at least one anionic polymer, and
about 0.5% to about 4% by weight of the starch,
wherein the weight percentages are based on the total weight of the composition.

9. The composition according to claim 1,
about 5% to about 20% by weight of the at least one colorant,
about 1% to about 12% by weight of the at least one anionic polymer,
about 0.5% to about 5% by weight at least one starch, and
about 1% to about 35% by weight at least one mattifying agent,
about 48% to about 91.5% by weight of the at least one auxiliary ingredient,
wherein the weight percentages are based on the total weight of the composition.

10. The composition according to claim 9,
about 5% to about 15% by weight of the at least one colorant,
about 2% to about 8% by weight of the at least one anionic polymer,
about 0.5% to about 4% by weight at least one starch, and
about 1% to about 25% by weight at least one mattifying agent,
about 25% to about 75% by weight of the at least one auxiliary ingredient,
wherein the weight percentages are based on the total weight of the composition.

11. The composition according to claim 1, wherein the viscosity of the composition is about 10 to 85 UD.

12. The composition according to claim 11 (provide narrower ranges), wherein the viscosity of the composition is about 25 to 60 UD.

13. The composition according to claim 1, wherein the Lab color space dimension L* is less than about 25 when the composition is dry.

14. The composition according to claim 13, wherein the Lab color space dimension L* is less than or equal to about 23.26 when the composition is dry.

15. The composition according to claim 1, wherein the Lab color space dimension L* is less than about 23 when the composition is wet.

16. The composition according to claim 15, wherein the Lab color space dimension L* is less than or equal to about 22.70 when the composition is wet.

17. The composition according to claim 1, comprising
at least one colorant, wherein the at least one colorant is iron oxide;
at least one anionic polymer, wherein the at least one anionic polymer is ethylene/acrylic acid copolymer; and
at least one starch, wherein the at least one starch is hydrolyzed corn starch,
wherein the at least one anionic polymer and the at least one starch forms hydrogen bonds to aid in pigment dispersion in the composition and to increase the viscosity of the composition.

18. The composition according to claim 17, comprising
at least one colorant, wherein the at least one colorant is iron oxide;
at least one anionic polymer, wherein the at least one anionic polymer is ethylene/acrylic acid copolymer; and
at least one starch, wherein the at least one starch is hydrolyzed corn starch;
at least one mattifying agent, wherein the at least one mattifying agent is wax;
at least one auxiliary ingredient, wherein the at least one auxiliary ingredient is film former;
wherein the at least one anionic polymer and the at least one starch forms hydrogen bonds to aid in pigment dispersion in the composition and to increase the viscosity of the composition.

19. A method of increasing color intensity of a color cosmetic composition, comprising:
(a) providing the composition according to claim 1;
(b) increasing an amount of the at least one pigment to a range from about 5% to about 15% by weight;
(c) reducing the at least one mattifying agent to a range from about 1% to about 25% by weight; and
(d) maintaining about 2% to about 8% by weight of the at least one anionic polymer and about 0.5% to about 4% by weight of the modified starch,
wherein the weight percentages are based on the total weight of the composition, and the composition exhibits increased color intensity.

20. A mascara comprising the composition according to claim 1.
